(19) United States
(12) Patent Application Publication
(10) Pub. No.: US 2023/0346254 A1
(43) Pub. Date: Nov. 2, 2023

(54) METHOD FOR MONITORING RESPIRATION OF A PATIENT DURING MOTION, AND A RESPIRATION MONITORING DEVICE

(71) Applicant: BABYMO SZOLGALTATO KFT., Szeged (HU)

(72) Inventors: Kata BENDE, Szeged (HU); Sándor KÓNYA, Gyula (HU); Balázs BENDE, Szeged (HU); Lívia BERKECZ-KOVÁCS, Szeged (HU); Árpád BEZDÁN, Szeged (HU); Zoltán CSÉPE, Csongrad (HU); Tamás KOVÁCS, Sandorfalva (HU); Tamás Zoltán LAUTNER, Kecskemet (HU); Zsolt TURCSÁNYI, Szolnok (HU)

(73) Assignee: BABYMO SZOLGALTATO KFT., Szeged (HU)

(21) Appl. No.: 17/924,932

(22) PCT Filed: May 12, 2021
(86) PCT No.: PCT/HU2021/050029

§ 371 Date: Nov. 11, 2022

(30) Foreign Application Priority Data
May 13, 2020 (HU) P2000160

(51) Int. Cl. A61B 5/08; A61B 5/0205; A61B 5/113; A61B 5/00

(52) U.S. Cl. CPC A61B 5/0816; A61B 5/0205; A61B 5/1135; A61B 5/746; A61B 5/683; A61B 5/6823

(57) ABSTRACT

A device and method for monitoring respiration of a patient is disclosed. The device includes a sensor unit provided with two V-shaped flex sensors and a measuring unit coupled to the sensor unit. The flex sensors can be resistive or capacitive. In use the measuring unit is attached to the chest of the patient and the sensor unit is attached to the abdominal wall of the patient. Relative displacement of the patient's chest and the abdominal wall are detected during respiration and patient's respiration rate is determined from the detected relative displacements of the chest and the abdominal wall.

The invention relates to a method for monitoring the respiration of a patient, in particular during movement and motion.

The invention also relates to a respiration monitoring device for carrying out the method according to the invention.

In today's fast-paced world, smart devices are part of our lives, we use them every minute of the day, we gather and analyze information, we take advice, we trust them and we can't imagine our lives without them. Among the achievements of this new age are wearable devices with sensors that we can wear to collect data about our own health, analyze it, look for trends, correlations between variables and draw conclusions about our health, our habits, and suggestions for our ideal conditions.

Various smart, wearable, sensor-based solutions are already available on the market to study our health, but they are still typically focused on a partial area such as ECG, heart rate, temperature measurement. Currently, there is no complex solution available on the market that would be able to monitor RR, HR, TEMP values integrated in a device, even during movement, and aggregate this data to display it to smart devices or to make recommendations to the wearer.

One of the unfortunate consequences of our times is the increase in disease, one of the reasons for which is mobility. Diseases that claim human lives are not linked to a geographical region, and viral pathogens that we have not encountered before, with no known course, no data and therefore difficult to control, are increasingly emerging, as is the case with coronavirus. Experts believe that these pandemic pandemics will become more frequent in the future, and that the role of wearable sensor solutions to monitor human vital signs will become even more important than before.

Various devices have long been used to monitor the physiological parameters of the human body, in particular the vital signs of patients and infants. Preserving the health of newborn babies is of greatest concern to parents. This is due both to the epidemics we are experiencing today and to the fatal diseases and disorders that specifically affect newborns and infants, such as cradle death or SIDS (Sudden Infant Death Syndrome). In response to the latter problem, so-called respiratory monitoring devices have been on the market for years, which monitor movement and sound an alarm if no breath is detected. The most common are breathing monitors that can be placed under the mattress in the baby's bed. One such solution is described in U.S. Pat. No. 9,693,730 (see FIG. 9). The main drawback of in-bed respiration monitoring devices is that they can only be used when the person is sleeping in the bed, i.e., they are not portable, and the design of the bed itself is not irrelevant. For example, they often do not work in beds with a grid, as they need to be placed on a stable base.

For those who want to keep their baby safe outside the cot, portable respiration monitors are now available. Patent US 2015/0305653A1, for example, discloses a breathing monitoring system for babies where the baby's breathing is monitored by a proximity sensor. Patent document WO2013/109892 discloses a method and system for monitoring the movement of body parts by measuring the user's body shape using a capacitive sensor. The solution consists of a sensor comprising two electrically conductive elements arranged in parallel and at a fixed distance from each other, and a controller connected to them. The conductive elements are applied to a flexible substrate. The displacement causes the parallel conductive elements to move closer to the user's body, thus reducing the measurable capacitance. In other words, in this solution, a change in capacitance is measured and the displacement of the user's body parts is deduced. An important feature of the sensor's measurement principle is that neither the conductive elements nor the controller is attached to the user's body, but are attached to the user's clothing.

The biggest disadvantage of known mobile respiration monitoring solutions is that they are sensitive to the baby's body position, so they can only be used reliably when the baby is at rest. Since false alarms are significantly increased when there is movement or motion (e.g., when travelling in a car, pushing a pram or sleeping with parents), these devices do not actually work reliably. Because of all these problems, many frustrated parents stop using the devices before the recommended age of 1 year, due to their limitations, or even do not start using them because they do not believe that they can provide real information about the child's condition.

The invention aims to provide a method and device free from the drawbacks of prior art solutions. In particular, it is an object of the invention to provide a method and device for monitoring the respiration of the subject not only at rest but also during movement or motion with the highest possible accuracy and with a minimum number of false alarms.

It is recognized that by using at least one flex sensor and its associated measuring unit, and by attaching them on the body in a special way according to the invention, monitoring of respiration beyond resting position can be achieved with higher accuracy and reliability than previous solutions in cases where the observed person is moving or being moved.

It is also recognized that during human breathing, there is always a relative distortion (relative displacement) of the abdominal wall and the chest, regardless of the person's body position, the state of movement or the type of breathing (abdominal or thoracic). It is recognized that measuring this relative displacement with a flex sensor capable of determining the deflection allows more reliable respiration monitoring than ever before when the person is moving or being moved.

The invention aims at providing a respiration monitoring method that is free from the disadvantages of state-of-the-art methods, i.e., that allows to reliably monitor the patient's breathing beyond resting conditions, both during movement and during motion, with the minimum false alarms.

It is also an object of the present invention to provide a respiration monitoring device which is free from the drawbacks of prior art devices, i.e., which allows the method according to the invention to be carried out.

The task according to the invention is achieved by a method for monitoring the respiration of a patient during movement and motion according to claim 1.

The task is further achieved by means of a respiration monitoring device according to claim 13.

The claimed task is solved by providing a respiration monitoring device comprising a sensor unit having at least one, preferably two flex sensors arranged in a V-shape, and a measuring unit coupled to the sensor unit, the respiration monitoring device being placed on the patient by attaching one unit of the respiration monitoring device to the patient's chest and the other unit to the patient's abdominal wall. The respiration monitoring device is used to detect the relative displacement of the patient's chest and abdominal wall during respiration by measuring the electrical signal provided by the flex sensors during respiration with the measuring unit and determining the respiration rate based on the detected relative displacements of the chest and abdominal wall.

The advantage of the method according to the invention is that it provides more reliable and accurate respiration monitoring than ever before, even when the person being monitored is moving or being moved.

Further preferred embodiments of the invention are defined in the dependent claims. Further details of the invention are described by means of embodiment examples and drawings. In the drawings

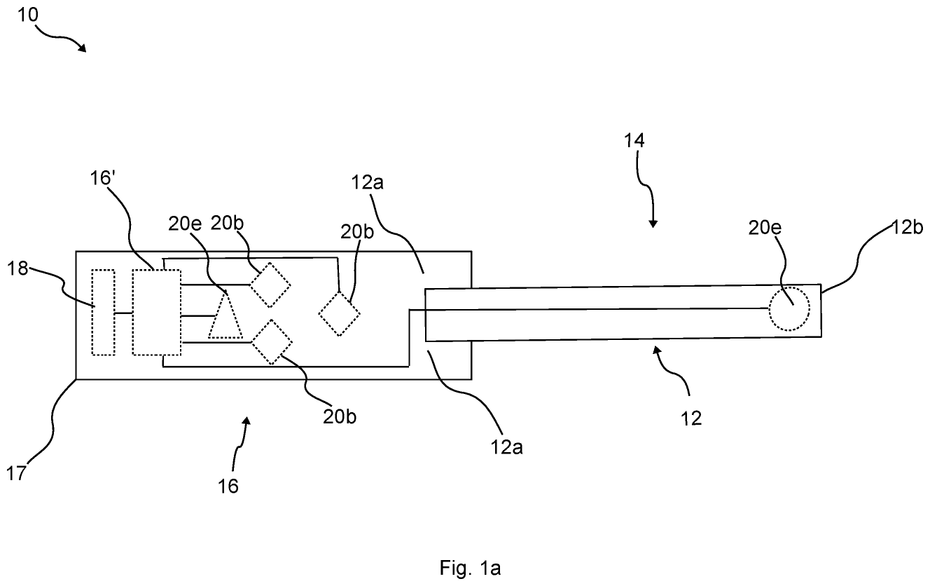

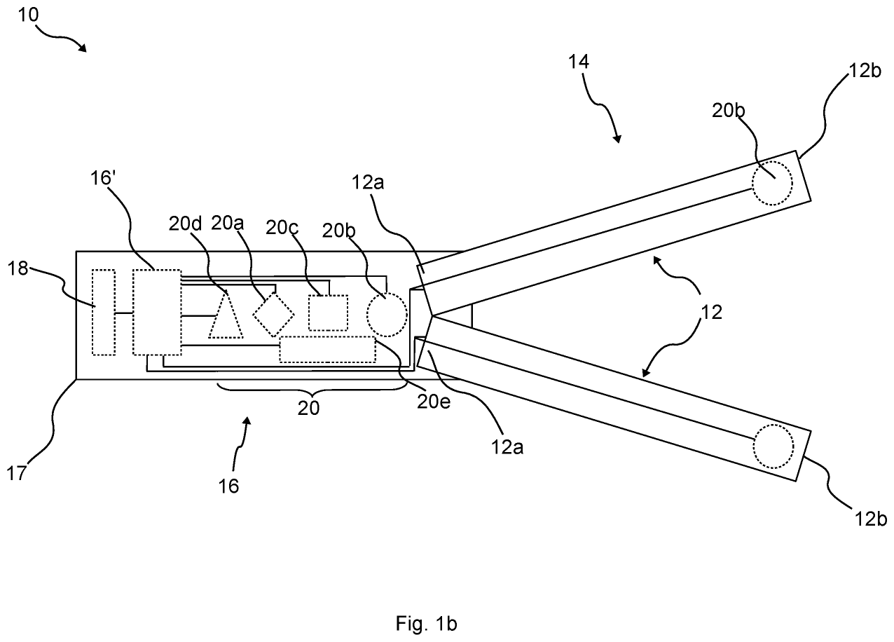

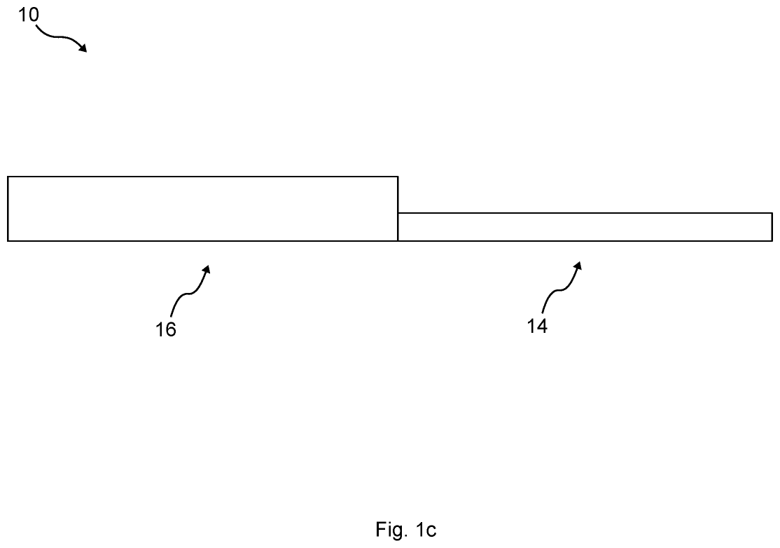

FIG. 1a is a schematic top view of an exemplary embodiment of a respiration monitoring device according to the invention comprising a single flex sensor,

FIG. 1b is a schematic top view of an exemplary embodiment of a respiration monitoring device according to the invention comprising two flex sensors arranged in a V-shape,

FIG. 1c is a schematic side view of the respiration monitoring device shown in FIG. 1b,

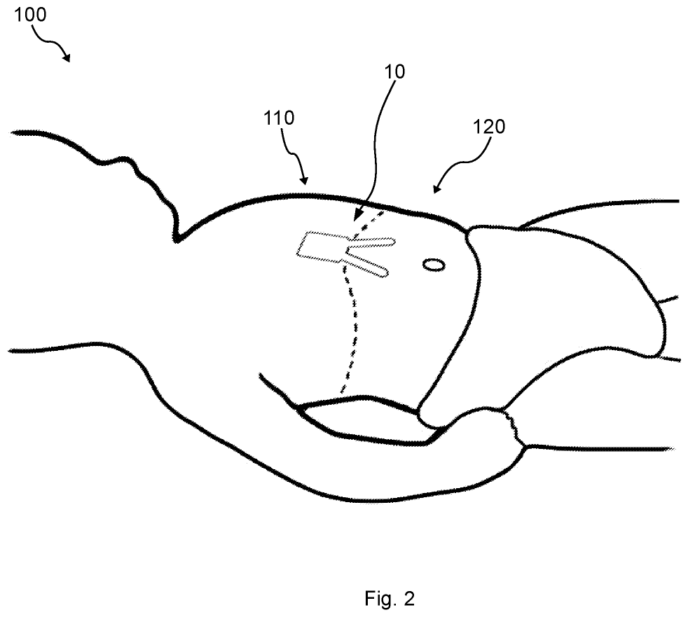

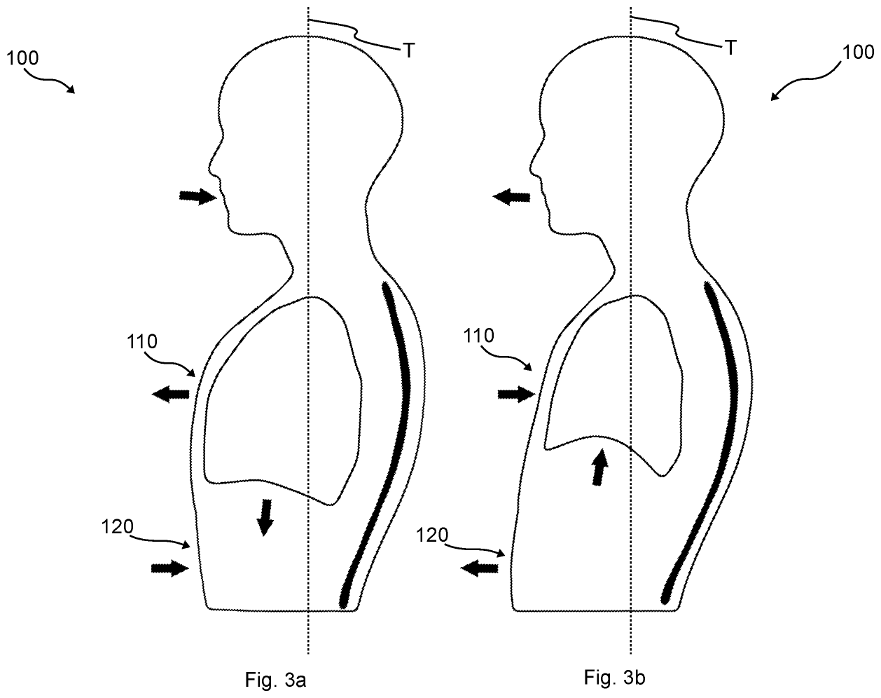

FIG. 2 is a schematic view showing the position of the respiration monitoring device attached to the patient's body during the method of the invention,

FIG. 3a is a schematic view of the patient's chest and abdominal wall in inhalation,

FIG. 3b is a schematic view of the patient's chest and abdominal wall in exhalation.

FIGS. 1a and 1b illustrate various exemplary embodiments of a respiration monitoring device 10 according to the invention. In the embodiment shown in FIG. 1a, the device 10 comprises a single flex sensor 12. The device 10 comprises a sensor unit 14 having at least one flex sensor 12, and a measuring unit 16 coupled to the sensor unit 14. In the context of the present invention, by flex sensor 12 is meant an electronic sensor of a flexible, easily bendable, preferably flat and elongated design, whose electrical signal changes in response to the bending of the flex sensor 12. The flex sensor 12 can be, for example, resistive, capacitive or any other sensor based on a principle known to the skilled person. In the particularly preferred embodiments shown in FIGS. 1a, 1b, the flex sensors 12 have an elongated shape and are connected to the measuring unit 16 via their ends 12a, for example by soldering. In the embodiment shown in FIG. 1b, the angle enclosed by the flex sensors 12 arranged in a V-shape is preferably an acute angle, but may also form a right angle or an obtuse angle. Preferably, the measuring unit 16 comprises a rigid or semi-rigid wall housing 17, for example made of plastic, which provides strength and protection for the measuring unit 16. The construction and function of the measuring unit 16 will be explained in more detail below.

In the method of the invention for monitoring the respiration of patients 100 during movement and motion the device 10 is provided as described above. It is noted that by patients 100 we mean primarily neonates, infants and young children, but the method according to the invention can of course be applied appropriately to adults. Movement of patients 100 in the context of the present invention is understood as changing the body position of patients 100 by their own efforts (e.g., limb movements, changing sleeping posture, etc.), while moving patients 100 is understood to mean changing position under external forces (e.g.,, travelling in a car, pushing a pram or sleeping with parents, etc.).

In the next step of the method according to the invention, the respiration monitoring device 10 is placed on the patient 100 such that one unit 14, 16 of the respiration monitoring device 10 is attached to the chest 110 of the patient 100 and the other unit 16, 14 is attached to the abdominal wall 120 of the patient 100, as shown, for example, in FIG. 2. In FIG. 2, the imaginary boundary separating chest 110 and abdominal wall 120 is illustrated by a dashed line. It is noted that a portion of the unit 14, 16 attached to the chest 110 may overlaps the abdominal wall 120 where appropriate; similarly, a portion of the unit 14, 16 attached to the abdominal wall 120 may overlaps the chest 110. The point, however, is that the units 14, 16 are secured to the patient 100 such that the movement of the unit 14, 16 secured to the chest 110 during respiration is substantially determined by the movement of the chest 110 and the movement of the unit 16, 14 secured to the abdominal wall 120 is substantially determined by the movement of the abdominal wall 120. That is, the unit 14, 16 attached to the chest 110 move with the chest 110, and the unit 16, 14 attached to the abdominal wall 120 move with the abdominal wall 120. In one possible embodiment, the units 14, 16 are attached directly to the skin of the patient 100 by adhesive, for example, by a skin-friendly adhesive commonly used in adhesive patches. The adhesive may for example be a double-sided skin-friendly adhesive strip, so that the strip can be easily replaced as the adhesive effect weakens. Note that the term attachment includes both full and partial fixation of units 14, 16. That is, the units 14, 16 may be attached along their entire surface facing the patient 100 to the chest 110 or abdominal wall 120, as the case may be, but embodiments where only a portion of the units 14, 16 are attached to the patient 100 (for example, only one half of the unit 16 and the ends of the flex sensors 12b) are also conceivable.

In the exemplary embodiment shown in FIG. 2, the measuring unit 16 is attached to the chest 110 of the patient 100 and the sensor unit 14 is attached to the abdominal wall 120 of the patient 100, but it is also possible to have the units 14, 16 attached in reverse, i.e., the measuring unit 16 is attached to the abdominal wall 120 and the sensor unit 14 is attached to the chest 110 (not shown). Note that, where appropriate, embodiments where the device 10 is attached to the patient 100 by means of a special garment or breathable strap are also conceivable. The method of the invention does not necessarily require direct contact (or contact via adhesive material) between the units 14, 16 and the skin of the patient 100, but in any case, the attachment should ensure that the units 14, 16 track the relative displacements of the chest 110 and abdominal wall 120 during respiration, i.e., that the units 14, 16 move with the chest 110 and abdominal wall 120 during respiration.

The relative displacements of the chest 110 and abdominal wall 120 of the patient 100 during respiration are illustrated in FIGS. 3a and 3b. As shown in FIG. 3a, during inhalation the chest 110 rises while the abdominal wall 120 sinks, and during exhalation the reverse is true, i.e., the chest 110 sinks and the abdominal wall 120 rises (see FIG. 3b). We recognize that this relative motion of the chest 110 and abdominal wall 120, essentially perpendicular to the longitudinal axis T of the patient 100, can be tracked by the device 10 regardless of the patient's 100 body position or the type of breathing (abdominal or thoracic).

In the next step of the method, detecting the relative displacement of the chest 110 and abdominal wall 120 of the patient 100 during respiration by means of the respiration monitoring device 10 in such a way that measuring with the measuring unit 16 the electrical signal provided by the at least one flex sensor 12 that is deflected during respiration. The measurement of the signal from the flex sensors 12 may be continuous or, where appropriate, intermittent with a predetermined sampling frequency, as will be apparent to the person skilled in the art. In one possible embodiment, the measuring unit 16 is attached to the chest 110 of the patient 100 and the sensor unit 14 is attached to the abdominal wall 120 of the patient 100. In this way, during inhalation, the measuring unit 16 starts to rise with the chest 110, while the unit 14 descends with the abdominal wall 120, so that the units 14 and 16 move parallel to each other, substantially perpendicular to the longitudinal axis T. Similarly, during exhalation, the measuring unit 16 starts to sink relative to the unit 14 (or the unit 14 starts to rise relative to the unit 16). The relative displacements of the units 14 and 16 will of course also occur if the measuring unit 16 is attached to the abdominal wall 120 and the sensor unit 14 to the chest 110. The relative displacements cause the at least one flex sensor 12 to deflect in one direction and then in the other direction as the breath is taken, both during inhalation and exhalation. The deflections cause the electrical signals measured by the flex sensor 12 to change periodically in accordance with the state of the breath cycle. For example, in the case of a resistive type flex sensor 12, the measured resistance will change, whereas in the case of a capacitive type flex sensor 12, the measured capacitance will change, as is obvious to the skilled person. The electrical signals from the at least one flex sensor 12 are received, processed and may be stored by the measuring unit 16. Accordingly, the unit 16 comprises an electronic central processing unit 16′ having a processor and/or a back-up storage medium for storing electronic data (e.g., memory card and/or RAM and/or ROM), as will be apparent to the skilled person. The electrical signal provided by the one or more flex sensor 12 may be either analog or digital, depending on the type of the flex sensor 12. In the former case, in a preferred embodiment, the signal from the flex sensor 12 is amplified in an analog manner and converted to a digital signal by means of an analog-to-digital converter (ADC) arranged in the measuring unit 16. In the embodiment with two flex sensors 12 arranged in a V-configuration, the signals from the flex sensors 12 are averaged using the measuring unit 16 and the averaged signal is processed. In this way, more accurate and reliable data on the respiration process can be obtained. An embodiment can also be envisaged whereby only the signal from one of the 12 flex sensors is used for the measurement in the baseline, but if the signal from this flex sensor 12 is lost (e.g., due to a failure), the signal from the other flex sensor 12 is used.

In the next step of the method of the invention, a respiratory rate (RR) is determined from the periodic variation of the electrical signals measured by the at least one flex sensor 12, based on the relative displacements of the chest 110 and the abdominal wall 120. As shown above, the change in the electrical signals measured by the at least one of the flex sensor 12 tracks the change in the relative position of the chest 110 and abdominal wall 120 during respiration one-to-one, so that each respiratory cycle can be identified by simply evaluating the electrical signals from one or more of the flex sensors 12 (e.g., by determining the peaks of the signal). As is well known, the instantaneous value of the respiratory rate can be determined by measuring the time elapsed between two successive breaths of the same phase of the cycle (e.g., the end of two successive inhalations). The respiratory rate can be expressed, for example, in breaths/minute. The respiratory rate can be determined from measured data using the measuring unit 16 or, may be, using an external device (not shown in the figures). In one possible embodiment, the electrical signal measured by the respiration monitoring device 10 is transmitted wirelessly to an external device (e.g., smartphone, computer) and the respiration rate is determined by the external device based on the data received. If the respiratory rate is determined using the unit 16, either the processed data containing the respiratory rate or (e.g., for further data analysis) the raw data is sent to the external device. Wireless data transmission can be done using, for example, known technologies such as Bluetooth and/or WiFi and/or LoRa (Long Range) as is obvious to the skilled person. The advantage of using an external device is that the measured respiratory rate can be made available to a person (e.g., doctor, parent, etc.) who is far away from the patient 100.

In a possible embodiment of the method according to the invention, one or more additional sensors 20 for measuring physiological parameters are provided in the sensor unit 14 selected from a group consisting of a pulse oximeter 20a, an ECG sensor 20b, a body temperature sensor 20c, an accelerometer 20d and a skin resistance sensor 20e, and the electrical signal of the one or more additional sensors 20 is measured by the measuring unit 16. The additional sensors 20 are arranged in the units 14 and/or 16 depending on their type. In a preferred examplary embodiment, one skin resistance sensor 20e is arranged in each of the units 14 and 16 in such a way as to provide contact between the skin of the patient 100 and the sensor 20e. The sensors 20e are then used to measure whether the units 14 and 16 are in contact with the skin of the patient 100. The one or more ECG sensors 20b may be provided in the unit 14 at the ends 12b of the flex sensors 12, but the unit 16 may also include an ECG sensor 20b. In a particularly preferred embodiment, the measuring unit 16 includes three ECG sensors 20b arranged in the measuring unit 16, preferably in a triangular configuration. In this case, there are no sensors 20b in the unit 14. The electrical signals from the sensors 20 may preferably also be transmitted wirelessly to an external device.

In a particularly preferred embodiment, if the electrical signal provided by the one or more of the flex sensors 12 is lost, the measuring unit 16 is used to check whether the signal provided by the one or more skin resistance sensors 20e is also lost. If so, a warning signal is sent by means of the respiration monitoring device 10. In the context of the present invention, the term “signal loss” of the one or more flex sensors 12 means that either no electrical signal is received from the one or more flex sensors 12 (the connection to the one or more flex sensors 12 has been lost) or the signal provided by the one or more flex sensors 12 is substantially constant (the periodic variation of the signals has ceased). The termination of the signal from the sensor 20e means that the given unit 14, 16 containing that sensor 20e has ceased to be in contact with the skin of the patient 100. In other words, the unit 14, 16 is no longer attached to the patient 100, rendering the device 10 unsuitable for measuring respiration rate. The warning signal sent by the device 10 may be audio and/or visual or a digital signal sent wirelessly to an external device. In the latter case, the external device may also provide an audio and/or visual warning signal.

In a preferred embodiment, wherein the device 10 comprises a pulse oximeter 20a and an ECG sensor 20b, the status of the electrical signal from the pulse oximeter 20a and the ECG sensor 20b is monitored independently of the status of the electrical signals provided by the at least one flex sensor 12. If the electrical signals from the pulse oximeter 20a and ECG sensors 20b cease, a check is made by the measuring unit 16 to determine whether the signal from the corresponding skin resistance sensor 20e has ceased, and if so, a warning signal is sent by the respiration monitoring device 10. Note that both the pulse oximeter 20a and the ECG sensor 20b can be arranged in units 14 and 16, respectively, so that the “corresponding” skin resistance 20e sensor means a sensor 20e arranged in the same unit 14, 16 as the pulse oximeter 20a or ECG sensor 20b, respectively. By the cessation of the electrical signal from pulse oximeter 20a and ECG 20b sensors, we mean that either no electrical signals are received at all (the connection with pulse oximeter 20a and ECG sensors 20b has ceased) or the measured signals are essentially stationary (the periodic variation of the signals has ceased).

In a particularly preferred embodiment, the method comprises defining a tolerance range of respiration rate limited by a minimum tolerance RR value and a maximum tolerance RR value, and a normal range of respiration rate limited by a minimum normal RR value and a maximum normal RR value within the tolerance range. In other words, the minimum normal RR value is larger than the minimum tolerance RR value and the maximum normal RR value is smaller than the maximum tolerance RR value. The maximum normal RR value is obviously greater than the minimum normal RR value and the maximum tolerance RR value is obviously greater than the minimum tolerance RR value. The unit of measurement of the RR values is the same as the unit of measurement of the respiratory rate. The RR values, i.e. the normal and tolerance ranges of respiratory rate they delimit, are preferably determined dynamically according to the age, possibly sex, of the patient 100 and preferably taking into account the current activity of the patient 100. For example, the normal range of respiratory rate for an average 4-month-old infant at rest (e.g., asleep) is 30-60 breaths/min, while for a 6-month-old infant the range is 24-30 breaths/min. Similarly, the normal range for the respiratory rate of a 4-month-old infant in an active state is 40-70 breaths/minute, and for a 6-month-old infant it is 30-40 breaths/minute. The status of the patient 100 can be determined by the signals from the additional sensors 20, such as the acceleration sensor 20d.

In a possible embodiment, the RR values are preferably stored in the measuring unit 16, so in this case the comparison of the currently measured respiratory rate values with the RR values is performed by the measuring unit 16. Note, however, that it is also possible to have an embodiment where RR values are stored on an external device and the unit 16 transmits the currently measured respiration rate values. In this case the comparison is performed by the external device receiving the data. If the respiratory rate determined by the device 10 falls outside the predefined normal range but within the tolerance range, a warning signal is sent by the respiration monitoring device 10. If the measured respiratory rate falls outside the tolerance range, an emergency signal, preferably different from the warning signal, is sent by the respiration monitoring device 10. The emergency signal may be audio and/or visual or a digital signal sent wirelessly to an external device. In the latter case, the external device may also send an audio and/or visual emergency signal.

In a preferred embodiment, the method according to the invention comprises monitoring the status of electrical signals from the pulse oximeter 20a and ECG sensor 20b independently of the status of electrical signals provided by the at least one flex sensor 12. Each of the pulse oximeter 20a and ECG sensors 20b is assigned a tolerance range limited by a minimum tolerance value and a maximum tolerance value, and a normal range limited by minimum normal values and maximum normal values within the tolerance ranges. The ranges are oxygen saturation (SpO2) and pulse ranges for pulse oximeter 20a and pulse ranges for ECG sensor 20b. Preferably, the normal and tolerance ranges assigned to each sensor are also chosen here according to the age, sex and preferably the current activity of the patient 100, for example based on literature data. For example, the normal range of the pulse assigned to the one or more ECG sensors 20b is 105-175 beats/min for an infant, and the tolerance range of the pulse is, for example, 90-190 beats/min as known to the skilled person. Preferably, the normal and tolerance values assigned to the pulse oximeter 20a and the normal and tolerance values assigned to the ECG sensor 20b are stored in the measuring unit 16. It is noted, however, that in some embodiments it is possible to have the normal and tolerance values stored on an external device and the unit 16 transmits the currently measured sensor signals to this external device. If the value of a physiological parameter measured by at least one of the pulse oximeter 20a and ECG sensors 20b falls outside the normal range assigned to the respective additional sensor 20, but within the tolerance range, a warning signal is sent by the respiration monitoring device 10. The warning signal may be audio and/or visual or a digital signal sent wirelessly to an external device. In the latter case, the external device may also send an audio and/or visual alarm. If the value of a physiological parameter measured by at least one of the pulse oximeter 20a and ECG 20b sensors falls outside the tolerance range assigned to the respective additional sensor 20, an emergency signal is sent by the respiration monitoring device 10.

In an exemplary embodiment, the status of the electrical signal from the body temperature sensor 20c is checked independently of the status of the electrical signals provided by the one or more flex sensors 12, and the body temperature sensor 20c is assigned a temperature tolerance range limited by a minimum tolerance value and a maximum tolerance value, and a temperature normal range limited by a minimum normal value and a maximum normal value within the tolerance range. If the temperature value measured by the body temperature sensor 20c falls outside the normal range but within the tolerance range, a warning signal is sent by the respiration monitoring device 10. The normal and tolerance ranges are preferably chosen according to the age, sex and current activity of the patient 100, as is apparent to the skilled person.

In one possible embodiment, the hydration of the skin is determined using a skin resistance sensor 20e, the state of the electrical signal from the sensor 20e is evaluated, and the sensor 20e is assigned a skin resistance tolerance range limited by a minimum tolerance value and a maximum tolerance value, and a skin resistance normal range limited by a minimum normal value and a maximum normal value within the tolerance range. If the skin resistance value measured by the skin resistance sensor 20e falls outside the normal range but within the tolerance range, a warning signal is sent by the respiration monitoring device 10. The value of the tolerance ranges is obvious to the skilled person.

The invention also relates to a respiration monitoring device 10 for carrying out the method according to the invention. The respiration monitoring device 10 comprises at least one sensor unit 14 with at least one flex sensor 12, and a measuring unit 16 coupled to the sensor unit 14 and adapted to receive and process electrical signals measured by the at least one flex sensor 12. In a preferred embodiment, the sensor unit 14 comprises two flex sensors 12 arranged in a V-shape. The one or more flex sensors 12 preferably have the elongated configuration shown in FIGS. 1a, 1b and preferably have a length of at least 2 cm. The length of the flex sensors 12 may vary depending on the body size of the patient 100, for example shorter flex sensors 12 may be preferred for infants and longer flex sensors 12 may be preferred for adults. Flex sensors 12 are preferably resistive flex sensors 12, but other types of flex sensors (e.g., capacitive) may be used.

In a particularly preferred embodiment, the device 10 includes one or more additional sensors 20 selected from a group consisting of a pulse oximeter 20a, an ECG sensor 20b, a body temperature sensor 20c, an accelerometer 20d, and a skin resistance sensor 20e for measuring physiological parameters. A preferred exemplary embodiment of the device 10 includes three ECG sensors 20b arranged in a triangular configuration in the measuring unit 16, as shown, for example, in FIG. 1a.

The respiration monitoring device 10 preferably comprises a communication sub-unit 18 for wireless transmission of the measured and processed sensor data, preferably arranged in the measuring unit 16. The sub-unit 18 may be, for example, a WiFi modem commonly used in portable electronic devices, or a Bluetooth chip integrated on a circuit, as is known to the skilled person.

In a particularly preferred embodiment, the device 10 comprises a power source, such as a rechargeable battery, preferably arranged in the measuring unit 16, for supplying power to the units 14 and 16.

Various modifications to the above disclosed embodiments will be apparent to a person skilled in the art without departing from the scope of protection determined by the attached claims

1. A method for monitoring respiration of a patient (100) during movement and motion, characterized by
providing a respiration monitoring device (10) comprising at least one sensor unit (14) with two flex sensors (12) and a measuring unit (16) operably coupled to the sensor unit (14),
placing the respiration monitoring device (10) on the patient (100) by attaching the measuring unit (16) to chest (110) of the patient (100) and attaching the sensor unit (14) to abdominal wall (120) of the patient (100),
detecting relative displacement of the chest (110) and the abdominal wall (120) during respiration by measuring an electrical signal provided by the flex sensors, and
determining respiration rate from detected relative displacements of the chest (110) and abdominal wall (120);
wherein the flex sensors (12) are selected from the group consisting of resistive flex sensor and capacitive flex sensor.

2. (canceled)

3. The method according to claim 1, characterized by the device (10) providing additional sensors (20) for measuring physiological parameters, selected from a group consisting of a pulse oximeter (20a), an ECG sensor (20b), a body temperature sensor (20c), an accelerometer sensor (20d) and a skin resistance sensor (20e), and measuring an electrical signal of the additional sensors (20) is measured by means of the measuring unit (16).

4. The method according to claim 3, characterized by checking by means of the measuring unit (16) whether a signal from a skin resistance sensor (20e) has ceased, and sending a warning signal by means of the respiration monitoring device (10) when said signal from the skin resistance sensor has ceased.

5. The method according to claim 3, characterized by checking the status of the electrical signal from the pulse oximeter (20a) and the ECG sensor (20b) independently of the status of the electrical signal from the at least one flex sensor (12), and, in the event of a cessation of the electrical signal from the pulse oximeter (20a) and ECG sensor (20b), a check is made by means of the measuring unit (16) to determine whether the signal from the one or more skin resistance sensors (20e) has ceased and, if so, sending a warning signal by means of the respiration monitoring device (10).

6. The method according to claim 1, characterized in that the flex sensors (12), define a respiration rate tolerance range limited by a minimum tolerance RR value and a maximum tolerance RR value, and a respiration rate normal range limited by a minimum normal RR value and a maximum normal RR value within the tolerance range and:
send a warning signal by means of the respiration monitoring device (10) when a measured respiratory rate is outside a normal range but within a tolerance range, and
send an emergency signal by the respiratory monitoring device (10) when a measured respiratory rate is outside a tolerance range.

7. The method according to claim 3, characterized in that the status of an electrical signal from the pulse oximeter (20a) and the ECG sensor (20b) is checked independently of the status of the electrical signals provided by the flex sensors (12), and that each of the pulse oximeter (20a) and ECG sensors (20b) is assigned a tolerance range limited by a minimum tolerance value and a maximum tolerance value, and a normal range limited by a minimum normal value and a maximum normal value within the tolerance range, and:
a warning signal is sent by means of the respiration monitoring device (10), when the value of a physiological parameter measured by at least one of the pulse oximeter (20a) and ECG sensors (20b) falls outside the normal range assigned to the respective additional sensor (20) but within the tolerance range, and
an emergency signal is sent by the respiration monitoring device (10) when a physiological parameter value measured by at least one of the pulse oximeter (20a) and ECG sensors (20b) is outside a tolerance range assigned to said additional sensor (20).

8. The method according to claim 3, characterized by checking the status of the electrical signal from the body temperature sensor (20c) independently of the status of the electrical signals provided by the at least one flex sensor (12), assigning to the body temperature sensor (20c) a tolerance range limited by a minimum tolerance value and a maximum tolerance value, and a normal range limited by a minimum normal value and a maximum normal value within the tolerance range, and sending a warning signal by the respiration monitoring device (10) when the physiological parameter value measured by the body temperature sensor (20c) falls outside the normal range but within the tolerance range.

9. The method according to claim 6, characterized in that the normal and tolerance ranges are determined dynamically according to age, sex and activity of the patient.

10. The method according to claim 1, characterized in that the respiratory monitoring device (10) is placed on the patient (100) by means of adhesive, a garment or a breathable strap.

11. The method according to claim 1, characterized in that a signal from the flex sensors (12) is amplified in an analogue manner and converted into a digital signal by means of an analogue-to-digital converter in the measuring unit (16).

12. The method according to claim 1, characterized in that a signal measured and processed by the respiration monitoring device (10) is transmitted wirelessly to an external device.

13. A respiration monitoring device (10) suitable for carrying out the method according to claim 1, characterized in that it comprises a sensor unit (14) provided with two flex sensors (12) arranged in a V-shape, and a measuring unit (16) coupled to the sensor unit (14) and adapted to receive and process electrical signals emitted by flex sensor (12), and wherein the flex sensors are selected from the group consisting of resistive flex sensors and capacitive flex sensors.

14. (canceled)

15. The respiration monitoring device (10) according to claim 13, characterized in that the flex sensors have an elongated shape and a length of at least 2 cm.

16. The respiration monitoring device (10) according to claim 13, characterized in that it comprises one or more additional sensors (20) selected from a group consisting of a pulse oximeter (20a), an ECG sensor (20b), a body temperature sensor (20c), an accelerometer sensor (20d) and a skin resistance sensor (20e) for measuring physiological parameters.

17. The respiration monitoring device (10) according to claim 16, characterized in that three ECG sensors (20b) are arranged in the measuring unit (16), preferably in a triangular shape.

18. The respiration monitoring device (10) according to claim 13, characterized in that it comprises a communication sub-unit (18) arranged in the measuring unit (16), which communication sub-unit (18) is adapted to wirelessly transmit data measured and processed by the respiration monitoring device (10).

19. (canceled)

20. The respiration monitoring device (10) according to claim 13, characterized in that it comprises a power source arranged in the measuring unit (16).

21. The respiration monitoring device (10) according to claim 20 wherein the power source is a rechargeable battery.
